SEQUENCE LISTING (1 sequence)

SEQ ID NO: 1 - DNA, 21 bp, Artificial Sequence
  Description of Artificial Sequence Oligonucleotide probe
tcgacagggactttccgagag

(19) United States
(12) Patent Application Publication
(10) Pub. No.: US 2004/0126385 A1
(43) Pub. Date: Jul. 1, 2004

(54) Methods and compositions for screening, diagnosing, or treating NF-kappaB related disease

(76) Inventors: Debajit K. Biswas, Newton, MA (US); Arthur B. Pardee, Brookline, MA (US)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/229,302

(22) Filed: Aug. 27, 2002

Related U.S. Application Data
(60) Provisional application No. 60/315,198, filed on Aug. 27, 2001.

(51) Int. Cl. C12Q 1/68; G01N 33/574; A61K 39/00

(52) U.S. Cl. 424/185.1; 435/6; 435/7.23

(57) ABSTRACT

A method of screening, diagnosing, or treating an NF-κB induced pathology through the use of NF-κB modulating agents. These cells isolated from pathological regions of a subject can be cultured in the presence or absence of a modulating agent to detect a change in proliferation of the cells, wherein a change in proliferation in modulator treated cells compared to control cells shows that the cells are derived from an NF-κB induced pathology. A subject that contains cells derived from an NF-κB induced pathology can be diagnosed with this pathology. NF-κB modulators can then be used to treat the NF-κB induced pathology.

### RELATED APPLICATIONS

This application claims priority to U.S. Ser. No. 60/315,198, filed Aug. 27, 2001, pending, which is incorporated by reference in its entirety.

### FIELD OF THE INVENTION

The present invention relates generally to methods and compositions for screening, diagnosing, or treating an NF-κB induced pathology through the use of NF-κB modulating agents. More specifically, it relates to methods for inhibiting growth of ER− cancer cells (including breast) by administering to the cells a composition which reduces NF-κB activity.

### BACKGROUND

The nuclear factor kappa B (NF-κB) is a collective nomenclature for the dimeric complexes composed predominantly of p50 and p65, 2 of a family of cellular proteins. NF-κB stimulates the expression of a spectrum of genes with diverging functions by binding to a specific DNA sequence. In most cell types, except B lymphocytes, NF-κB exists in the cytoplasm in an inactive state being complexed with an inhibitory protein called IκB. The activation of NF-κB is initiated by specific signals generated by interaction of activators with respective receptors which are then transmitted via a cascade of interacting kinases that ultimately activate IκB-kinase (Ikk) followed by phosphorylation and degradation of IκB. The active complex is then translocated into the nucleus and stimulates the expression of responsive genes. The multifaceted gene regulatory properties qualify NF-κB as one of the pivotal cellular regulatory molecules that are essential for the maintenance of homeostasis and physiology of multicellular organisms.

Although the role of the NF-κB family of proteins in immune, inflammatory, and apoptotic responses has been well documented, the role of NF-κB in tumorigenesis is not as well documented. The present invention is related to the discovery disclosed herein that the level of active NF-kB plays a role in tumorigenesis, more specifically in ER− cancers, including ER− breast cancers. The present invention also relates to NF-kB as a target for therapy for this class of hormone-dependent tumors.

The class of tumors called “hormone-dependent” tumors generally occur in the target organs of hormones, and their growth can be promoted by or dependent on the presence of those hormones. For example, the growth of some mammary cancers is promoted by estrogen, the growth of some prostate cancers is promoted by androgen, and the growth of some thyroid cancers is promoted by thyroid stimulating hormone. Hormonal endocrine therapies are widely used for the treatment of such hormone-dependent tumors. For example, excision of estrogen-producing ovaries has been employed as an endocrine therapy for some estrogen-dependent mammary cancers. In addition, a widely used treatment for mammary cancer is the administration of an anti-estrogenic agent such as tamoxifen, which competes with estrogen for binding to the estrogen receptor (ER), thereby exerting an antitumor effect. A biopsy of a mammary cancer is generally examined for the presence or absence of estrogen receptors in the cancerous tissue, in order to determine whether administration of an anti-estrogenic agent is indicated. Current methods utilize immunodetection of the ER to determine the presence or absence of ER.

If the presence of ER is demonstrated by radiolabeled ligand binding or immunodetection, then current therapies indicate administration of anti-estrogenic agents. Anti-estrogenic agents are those compounds which compete with estrogen for binding to estrogen receptors. Selective estrogen receptor modulators (SERMs) exhibit a pharmacologic profile characterized by estrogen agonist activity in some tissues with estrogen antagonist activity in other tissues. These compounds were initially called “anti-estrogens,” but it was subsequently recognized that this inadequately described their spectrum of activities. The first widely used SERM, tamoxifen, has estrogen antagonist activity in breast tissue but shows estrogen-like activity in other tissues. An unwanted effect of tamoxifen was its estrogen-like action on the endometrium. Second-generation compounds have since been developed, most notably raloxifene, which has estrogen-like actions on bone, lipids and the coagulation system, and estrogen antagonist effects on the breast and uterus. The above-described compounds are only effective, however, in mammary cancers in which the ER is present, as well as functional.

The current therapeutic approach with antihormones, targeted at hormone receptors, is effective only in a fraction of breast cancer patients. All estrogen receptor negative (ER−) and also a fraction of ER positive (ER+) tumors do not respond to antihormone treatment. Thus, alternative treatment protocols aimed at different targets for these classes of antihormone nonresponsive breast cancers need to be explored.

We demonstrate herein that the level of active NF-kB plays a role in tumorigenesis, more specifically in ER− cancers, and also disclose that NF-kB is a target for therapy for this class of human breast cancers. Currently there is very limited therapy for ER− breast cancers, so that there is a need in the art for such therapy. Compositions and methods for such needed treatment methods are described herein, including the use of compounds which inhibit NF-κB activation.

### SUMMARY OF THE INVENTION

In one aspect, the invention provides a composition, which, when administered to mammalian subjects with ER− cancer, selectively inhibits activation of NF-κB and results in regression of ER− tumor cell growth in the mammalian subjects. The composition can also be in the form of a pharmaceutical composition or in a kit.

Another embodiment of the invention is a method for inhibiting growth of ER− breast cancer cells, the method comprising administering to the cells a composition which reduces NF-κB activity in the cell in an amount sufficient to inhibit growth of the ER− breast cancer cells. The method can be carried out on mammalian cells, including human cells, and can be carried out in vitro or in vivo.

Another embodiment of the invention is a method for diagnosing and treating mammalian cancers in a subject, the method comprising obtaining cancer cells from the subject; testing the cancer cells from the subject for the presence of estrogen receptor; diagnosing the mammalian cancer as ER+ if estrogen receptor is present in the cells or as ER− if estrogen receptor is absent from the cells; and administering to the subject diagnosed with ER− cancer a composition which reduces NF-KB activity, in an amount sufficient to inhibit growth of ER− breast cancer cells. The composition used in this method can be kinase inhibitors that are involved in the activation of NF-κB, similar to the Go compounds, and even more preferably Go6796.

Another embodiment of the present invention is a method for treating ER− cancers in a mammalian subject, the method comprising administering to the mammal a composition which reduces NF-κB activity and monitoring the mammal to determine the state of the cancer, wherein the composition is administered in an amount sufficient to inhibit the growth of ER− breast cancer cells. The composition used in this method can be a kinase inhibitor, more preferably a Go compound, and even more preferably Go6796.

Another embodiment of the present invention is a method of treating an NF-κB mediated pathology in a subject wherein the pathology is treated by administration of Go6976 to the subject. The pathology treated can be a cancer, more preferably a breast cancer, and yet more preferably ER− breast cancer. In this embodiment, the subject treated can be mammalian, and more preferably human. Go6976 can be administered by any means known to one skilled in the art, including oral administration, intraperitoneal administration, injective administration, suppository administration, and transdermal administration.

Another embodiment of the present invention is a method for diagnosing patients who would be receptive to treatment with a composition, which, when administered to mammalian subjects with ER− cancer, selectively inhibits activation of NF-κB and results in regression of ER− tumor cell growth in the mammalian subjects. The method comprises obtaining cells from the patient; testing the cells for the presence of estrogen receptor; and testing the cells for the presence of activated NF-κB; wherein the absence in the cells of estrogen receptor in combination with the presence in the cells of activated NF-κB indicates a patient who would be receptive to treatment.

In yet another embodiment, the present invention is directed to a method for identifying a potential therapeutic agent for use in the treatment of ER− breast cancers, the method comprising providing ER− cells, tissues, or animals; contacting the ER− cells, tissues, or animals with a composition comprising a candidate substance, wherein the candidate substance inhibits NF-κB activity; and monitoring the progression of the ER− cancer; wherein, if the progression of the ER− cancer is reduced, the candidate substance is identified as a potential therapeutic agent.

In an additional embodiment, the present invention is directed to a method of screening for cancer cells in a tissue, the method comprising isolating the tissue; splitting the tissue into a first portion and a second portion; and culturing the first portion with Go6976 and culturing the second portion without Go6976; wherein if the tissue of the first portion decreases in proliferation as compared to the second portion, the tissue contains cancer cells with activated NF-κB. In another embodiment, the cancer cells have NF-κB activity. Additionally, the tissue can be mammalian tissue, more preferably human.

In another embodiment, the invention is directed to a method for detecting NF-κB activity in a cell culture, the method comprising isolating the cell culture; splitting the cell culture into a first portion and a second portion; and culturing the first portion with Go6976 and culturing the second portion without Go6976, wherein if the cell culture of the first portion decreases in proliferation as compared to the second portion, the cell culture has NF-κB activity. In this embodiment, the cell culture can be derived from epithelial cells, more preferably from mammary epithelial cells.

In another embodiment, the present invention is directed to a method of diagnosing the type of cancer cells comprising a tumor in a subject, the method comprising isolating the tumor from the subject; splitting the tumor into a first portion and a second portion; and culturing the first portion with Go6976 and culturing the second portion without Go6976,wherein a decrease in proliferation of the tumor of the first portion in relation to the tumor of the second portion diagnoses one type of cancer cell from another type of cancer cell. The types of cancer cells being diagnosed can be estrogen receptor negative breast cancer and estrogen receptor positive breast cancer. In a preferred embodiment, the diagnosis of the cancer cell types is dependent upon differential NF-κB activity.

In still another embodiment, the present invention is directed to a method of screening for a NF-κB mediated pathology in a tissue, the method comprising isolating the tissue; splitting the tissue into a first portion and a second portion; and culturing the first portion with Go6976 and culturing the second portion without Go6976; wherein if the tissue of the first portion decreases in proliferation as compared to the second portion, the tissue has an NF-κB mediated pathology. In this embodiment, the NF-κB mediated pathology can be cancer, more preferably breast cancer. Additionally, the tissue can be mammalian tissue, more preferably human tissue.

These and other aspects of the invention will be evident upon reference to the following detailed description and attached drawings. Additionally, various references are set forth herein which describe in more detail certain aspects of the invention, and which are hereby incorporated by reference in their entirety.

### BRIEF DESCRIPTION OF THE DRAWINGS

FIG. 1. Inhibition of growth of tumors by Go6976. Tumors were generated in female A-J mice by implantation of CSMLO cells (10⁶) on day 0 (indicated by the arrow). Go6976 (0.5 ml of 0.05 mM solution in 0.1% DMSO) was administered 48 h later to a group of five animals, twice a week locally at the site of implantation of cells (♦). Control group (five animals) received 0.5 ml of 0.1% DMSO similarly (□). Growth of tumors was monitored by measurements of tumor volume at regular intervals (32).

FIG. 2. Regression of tumors by Go6976. (A) CSMLO cells were implanted on day 0 (indicated by the arrow) as for FIG. 1. Go6976 was administered on day 21 (indicated by the arrow), locally under the tumors of the five animals with comparatively larger tumors (▪). Another five animals received the same volume of 0.1% DMSO (♦) on the same day, and the control group of five animals received nothing (□). (B) One representative of five animals from each group. Animals 1 and 2 are DMSO- and Go6976-treated non-tumor-bearing animals, respectively. Animal 3 is tumor-bearing and without treatment. Animals 4 and 5 represent tumor-bearing groups treated with DMSO and Go6976, respectively. Animal 6 is an A-J mouse that received nothing.

FIG. 3. Active NF-κB complex in CSMLO cells: Stimulation by PMA and inhibition by Go6976. (A) Nuclear extracts (5 μg protein) from control and PMA-treated (20 ng/ml for 18 h) CSMLO cells were incubated in a standard EMSA reaction mixture containing [γ-³²P]-labeled double-stranded oligonucleotide plus Go6976 at the indicated μM concentrations for 48 h and subjected to nondenaturing PAGE (7, 31). The autoradiographic signals of the retarded NF-κB-[³² P]DNA complex is indicated by the upper arrow and the free [γ-³²P]-labeled probe by the lower arrow. (B) The NF-κB-[³²P]DNA complex was characterized by supershift assay with anti-p50 (lanes 3 and 4) or p65 (B, lanes 5 and 6) antibodies. Nuclear extracts were incubated with specific antibodies for 15 min at room temperature, followed by incubation for an additional 30 min in the presence of [γ-³²p] double-stranded NF-κB oligonucleotide, and subjected to EMSA as described (7, 31). The supershifted complexes are indicated by the upper arrow.

FIG. 4. Histology of tissues from untreated and Go6976-treated tumor-bearing animals. Tumor growth and treatment conditions are the same as described for FIG. 1. Tumor, liver, and lung tissues from untreated and treated tumor bearing animals were dissected 11 days after the initiation of treatment and 32 days after implantation of the cells. Tissues were processed for hematoxylin/eosin (H&E) staining, examined under a light microscope, and photographed at the indicated magnifications. Arrows show mitotic cells in untreated tumor tissue. Residual tumor (T) and necrotic cells (N) of the treated tumor are shown. Stars in the treated block indicate pycnotic cells with apparent fragmentation and clumping of nuclear DNA. Treated and untreated liver and lung tissues did not show significant microscopically detectable damages and were not different from liver and lung tissues of normal mice without tumors (not shown).

FIG. 5. Inhibition of NF-κB activation and down stream events by stable expression of dominant-negative IκB-kinase β (dnIkkβ). (A Upper) dnIkkβ-Expressing transfectant cells by light microscopy (i), 4′,6-diamidino-2-phenylindole (DAPI)-stained nuclei (ii), and immunofluorescence with anti-FLAG antibody in the presence of the secondary antibody (iii). The positive signals show dnIkkβ-conjugated FLAG protein in the cytoplasm. (Lower) Processed vector-control plasmid-transfected CSMLO cells in which no FLAG protein could be detected (vi). (B Upper) Active NF-κB was determined by its [γ-³²P]DNA binding activity by EMSA, in three dnIkkβ-expressing stable transfectants (dnIkkβ1-1, dnIkkβ1-3, and dnIkkβ1-5) and (Lower) in parent CSMLO cells and vector control plasmid expressing transfectant (vect1-5). (C Upper) The level of ccD1 in the same three-dnIkkβ-expressing CSMLO and vector control transfected cells, as measured by Western blot analysis. (Lower) Actin analyzed similarly by immunodetection with anti-β-actin antibody that serves as a loading control.

FIG. 6. Loss of tumorigenic potential of CSMLO cells by the stable expression of pdnIkkβ in A-J mice. Tumors were generated in female A-J mice by implanting either CSMLO cells (five animals), or two vector control plasmid transfected clones (vect1-3 and vect1-5, three animals per clone) or two pdnIkkβ-expressing clones (pdnIkkβ1-1 and pdnIkkβ-1-3, three animals per clone). Average with standard deviations of CSMLO-administered animals (♦), vector-control-administered animals (▪, six animals), and one of the two pdnIkkβ-expressing-clones (pdnIkkβ1-3)-administered animals (Δ, three animals) are plotted. The other pdnIkkβ1-1 clone did not form any tumor even 32 days after implantation of the cells in any one of the three animals (ρ).

### DETAILED DESCRIPTION OF THE INVENTION

The present invention relates generally to methods and compositions for screening, diagnosing, or treating an NF-κB induced pathology through the use of NF-κB modulating agents. The NF-κB induced pathologies which can be diagnosed and treated include a variety of cancers. One specific NF-κB induced pathology which can be treated with compositions which inhibit activation of NF-κB is estrogen receptor negative cancer (ER−) including breast cancer. The current breast cancer therapeutic approach, treatment with antihormones which are targeted at hormone receptors, is effective only in a fraction of breast cancer patients. All estrogen receptor negative (ER−) and also a fraction of ER positive (ER+) tumors do not respond to antihormone treatment. Thus, alternative treatment protocols aimed at different targets for these classes of antihormone nonresponsive breast cancers need to be explored. One such alternative treatment protocol is treatment with compositions which inhibit activation of NF-κB.

Breast cancer patients have been classified as either estrogen receptor positive (ER+), where ER is present, or ER negative (ER−), wherein ER is absent. The presence or absence or ER in a patient is currently determined by immunodetecting the receptor with a specific antibody.

Currently, the level of estrogen receptor (ER) present in a patient is the main determinant for the therapeutic management of ER(+) breast cancer patients. Current therapies are aimed at interrupting the role which ER plays in regulating cellular proliferation. ER is a member of the steroid family of receptors, which are hormone activated transcription factors. ER is present in cells as an inactive complex associated with the inhibitory heat shock protein hsp90. Binding of estrogen to the receptor releases this inhibitory heat shock protein and initiates a series of downstream events resulting in overexpression of genes responsible for enhanced and uncontrolled growth of breast cancer cells. Growth of many human breast cancers is therefore regulated by estrogen (E2) and progesterone (Pr). Generally, the ER in ER (+) breast cancer patients is targeted for therapy with anti-hormones, such as tamoxifen and raloxifene. Compounds such as tamoxifen (TAM) and raloxifene bind to ER and cannot confer the active configuration to the receptor in mammary epithelial cells, thereby blocking the transmission of E2-ER initiated signals for cell proliferation. These compounds are thus designated as anti-hormones (or SERMs) and are candidates for breast cancer therapy. Their therapeutic activity is limited to ER (+) breast cancers, since their mechanism of action involves competing with estrogen for binding to ER, and altering ER so that it can no longer cause cellular proliferation.

The therapeutic activity of antihormones should be effective for all ER(+) breast cancers. However, only approximately 60% of ER(+) patients respond to tamoxifen, raloxifene, and other antihormone therapies. The remaining ER(+) and all ER negative (ER−) breast cancers constitute a major fraction of breast cancers which do not respond to anti-hormone therapy. Accordingly, a need exists for treatment of ER− breast cancers.

The present invention is directed to compositions and methods for treating such ER− cancers (including breast cancers) by administering compositions which reduce the level of nuclear factor kappa-B (NF-kB) in an amount sufficient to inhibit growth of ER− breast cancer cells. The level of NF-kB has been shown to be elevated in ER− human breast cancers, as compared with ER+ cells. This could be correlated with the increased level of epidermal growth factor family receptors (EGFR) in ER− cells. Our previous results have demonstrated that activation of NF-kB is a downstream consequence of EGF-EGFR interaction (7). A pathway has been proposed for the EGF-EGFR-mediated cell proliferation signal that involves activation of phosphatidylinositol 3-kinase (PI3-kinase), protein kinase C, and NF-kB with overexpression of the downstream cell cycle regulatory protein cyclin D1 (ccD1) and retinoblastoma (Rb) phosphorylation (7). These results, along with its anti-apoptotic action, strongly suggest the involvement of activated NF-kB in ER− breast cancers (5, 7-10). This role is examined here with a mouse tumor model generated with an ER− mouse mammary epithelial carcinoma cell line, CSMLO (11, 12). See Examples 1-8 herein.

NF-kB is a transcription factor which regulates a gene expression. It is known in the art that NF-κB is specific to B-lymphocytes (B-cells) and also to be B-cell stage specific. NF-κB was originally detected because it stimulates transcription of genes encoding kappa immunoglobulins in B lymphocytes. It has subsequently been shown that transcription factor NF-κB, previously thought to be limited in its cellular distribution, is, in fact, present and inducible in many, if not all, cell types and that it acts as an intracellular messenger capable of playing a broad role in gene regulation as a mediator of inducible signal transduction. It has been demonstrated that NF-κB has a central role in regulation of intercellular signals in many cell types. For example, NF-κB has been shown to positively regulate the human β-interferon gene in many, if not all, cell types. Additionally, it is clear not only that NF-κB is not tissue specific in nature, but also that in the wide number of types of cells in which it is present, it serves the important function of acting as an intracellular transducer of external influences.

Additionally, genes which are considered to be subjected to expression regulation by NF-kB are often those participating in immunity inflammation reactions such as inducible nitric oxide synthase (iNOS), inflammatory cytokines such as TNF-.alpha., IL-1, IL-6 and IL-8, and cell adhesion molecules such as ICAM-1, VCAM-1 and ELAM-1 (Collins, T., Read, M. A., Neish, A. S., Whiteley, M. Z., Thanos, D. and Maniatis, T. (1955) Faseb. J., 9, 899-909). Moreover, when an inflammatory cytokine binds to its receptor, the cytokine is known to transduce a signal which activates NF-kB through various routes, which is considered to aggravate the inflammation. The activation of NF-kB is understood to be a cause and an exacerbation factor of a variety of diseases (Baeuerle, P. A. and Baichwal, V. R. (1997) Adv. Immunol., 65, 111-137).

Furthermore, it has been reported that HIV, HTLV-1, CMV, adenovirus, or the like activates NF-kB in the host cells (Dezube, B. J., Pardee, A. B., Beckett, L. A., Ahlers, C. M., Ecto, L., Allen-Ryan, J., Anisowicz, A., Sager, R. and Crumpacker, C. S. (1992) J. Acquir. Immune Defic. Syndr., 5, 1099-1104, Nabel, G. and Baltimore, D. (1987) Nature, 326, 711-713, Fazley, F., Dezube, B. J., Allen-Ryan, J., Pardee, A. B. and Ruprecht, R. M. (1991) Blood, 77, 1653-1656, Munoz, E. and Israel, A. (1995) Immunobiology, 193, 128-136). Activation of NF-kB increases the transcription, proliferation and infectivity of the virus.

Furthermore, activation of NF-kB has been confirmed in various chronic inflammatory diseases (Marok R., Winyard P G, Coumbe A, Kus M L, Gaffney K, Blades S, Mapp P I, Morris C J. Blake D R, Kaltschmidt, Baeuerle P A (1996) Arthritis Rheum. 39, 583-591).

As described above, the role of the NF-kB family of proteins in immune, inflammatory, and apoptotic responses is well documented (6, 13, 14). These are activated by growth factors, cytokines, and mitogens that control cell proliferation, differentiation, and morphogenesis and are transcription factors that activate several cell cycle regulatory proteins (13-17). The role of NF-kB in tumorigenesis has also been demonstrated herein, by higher levels of activated NF-kB in ER− tumor cells. NF-kB exists in an inactive state in most cell types, except B lymphocytes (16). Activation of NF-kB involves phosphorylation of two conserved serines in the N-terminal domain of I kB, which is then degraded by the ubiquitin pathway (18). Signaling of NF-kB activation is a multistep process transmitted by a cascade of kinases leading to activation of the ultimate kinase complex, Ikk, composed of Ikk-a, Ikk-b, and the regulatory protein Ikk-g (also known as NEMO) (19-21). Different NF-kB activating agents generate diverging signals that ultimately activate Ikk by regulating the function of one of these components. In general, Ikk-b has a much higher level of kinase activity than Ikk-a and plays a critical role for the degradation of I kB and consequently the activation of NF-kB (22, 23). Thus, the Ikk complex is a potential target for controlling NF-kB activation and its functions.

For example, mice deficient in either Ikk-a, Ikk-b, or both exhibit multiple developmental and morphological defects and enhanced apoptosis leading to embryonic lethality or death at birth that could be correlated to lack of NF-kB activation (24, 25). Enhanced apoptosis in liver causing embryonic lethality observed in Ikk-b-deficient mice could be related to tumor necrosis factor (TNF) signals, because it is overcome in progeny of mating to TNF-null mice (26).

Although a substantial amount of work is done with genetically altered animals leading to stable loss of activation of NF-kB and its consequences, very limited experiments have been done with externally introduced agents that selectively inhibit NF-kB activation. The anti-inflammatory activity of a peptide that specifically inhibited the interaction of Ikk-g with Ikk complex and selectively blocked activation of NF-kB is demonstrated in an animal model (27).

We demonstrate here in a mouse tumor model the antitumorigenic activity of a compound that inhibits activation of NF-κB without causing significant detectable cellular damage of vital organs. Furthermore, selective activation of NF-κB by stable expression of a dnIkkβ mutant plasmid induced loss of tumorigenic potential of the parent CSMLO cells, thus strongly suggesting a role of this transcription factor in ER− mammary epithelial cell carcinogenesis. These results are discussed in more detail in the examples.

Compositions

As discussed above, in one aspect, the invention provides a composition, which, when administered to mammalian subjects with ER− cancer, selectively inhibits activation of NF-κB and results in regression of ER− tumor cell growth in the mammalian subjects. The composition can also be in the form of a pharmaceutical composition or in a kit.

The compositions of the invention can be incorporated into pharmaceutical compositions suitable for administration. Such compositions typically comprise the substance that inhibits NF-κB activation and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., the substance that inhibits NF-κB activation) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The pharmaceutical compositions can be included in a container, pack, or dispenser together with instructions for administration.

Methods for Diagnosing and Treating Mammalian Cancers

Another embodiment of the invention is a method for inhibiting growth of ER− cancer cells, the method comprising administering to the cells a composition which reduces NF-κB activity in the cell in an amount sufficient to inhibit growth of the ER− cancer cells. The method can be carried out on mammalian cells, including human cells, and can be carried out in vitro or in vivo.

Suitable in vitro or in vivo assays can be performed to determine the effect of a composition which reduces NF-κB activity and whether its administration inhibits growth of ER− cancer cells. In various specific embodiments, in vitro assays may be performed with representative ER− cancer cells, to determine if a given therapeutic exerts the desired effect upon the cell type(s). Compounds for use in therapy may be tested in suitable animal model systems including, but not limited to rats, mice, cows, monkeys, rabbits, and the like, prior to testing in human subjects. Similarly, for in vivo testing, any of the animal model system known in the art may be used prior to administration to human subjects.

Another embodiment of the invention is a method for diagnosing and treating mammalian cancers in a subject, the method comprising obtaining cancer cells from the subject; testing the cancer cells from the subject for the presence of estrogen receptor; diagnosing the mammalian cancer as ER+ if estrogen receptor is present in the cells or as ER− if estrogen receptor is absent from the cells; and administering to the subject diagnosed with ER− cancer a composition which reduces NF-KB activity, in an amount sufficient to inhibit growth of ER− cancer cells. The composition used in this method can be a kinase inhibitor, more preferably a Go compound, and even more preferably Go6796.

Another embodiment of the present invention is a method for treating ER− cancers in a mammalian subject, the method comprising administering to the mammal a composition which reduces NF-κB activity and monitoring the mammal to determine the state of the cancer, wherein the composition is administered in an amount sufficient to inhibit the growth of ER− cancer cells. The composition used in this method can be a kinase inhibitor, more preferably a Go compound, and even more preferably Go6796.

Another embodiment of the present invention is a method of treating an NF-κB mediated pathology in a subject wherein the pathology is treated by administration to the subject of a compound which inhibits activation of NF-κB. NF-κB mediated diseases include a variety of types of cancer, including breast cancer, colon cancer, and prostate cancer. Evidence that activated NF-κB is involved in cancers is based at least the following: i) NF-kB proteins are member of the proto-oncogene family; ii) NF-kB 2 gene and Bcl-3 gene are translocated in lymphomas; and iii) NF-kB is activated by viral transforming proteins. See A. S. Baldwin, Jr. Ann. Rev. Immunol, 14: 649-681, 1996.

NF-κB mediated diseases also include a variety of immune diseases, including arthritis and Ataxia Telangiectasia. The role of NF-kB in arthritis is based on at least the following: i) it is activated in synovium; and ii) prednisone and gold that are used for treatment of arthritis also block NF-kB activation. See, Yang J, Martin, J., Kano, T, Kitade Y, 1995, Okamoto, T. FEBS letters. 361: 89-96. NF-kB is activated in Ataxia Telangiectasia, a human disease associated with immunological deficiencies. See Jung, M., Zhang, Y, Lee, S, Drischilo, A 1995; Science 268: 1619-1621.

Additionally, NF-κB mediated diseases include a variety of inflammatory-based pathologies, including artherosclerosis. It has been shown that activated NF-kB is involved in the inflammatory process when oxidized lipids become trapped in the extracellular matrix of the subendothelial space. See Berliner, J, Navab, M., Fogleman, A., Frank, J., Demer, L., Edwards, P., Watson, A, ,Lusis, A, 1995Circulation 91: 2488-2496. Additionally, physical stress such as irradiation also induces activation of NF-kB and related genes iNOS and COX 2. See Pahl, L. L. Oncogene 18: 6853-6866, 1999. Accordingly, the NF-κB mediated pathology to be treated can be an inflammatory-based pathology, an immune-based pathology, or a type of cancer, more preferably a breast cancer, and yet more preferably ER− breast cancer. In this embodiment, the subject treated can be mammalian, and more preferably human.

Substances that inhibit the activation of NF-κB include kinase inhibitors, more specifically Go compounds, and most specifically Go6796. Representative kinase inhibitors include the IKK-activity inhibitors Silibinin, Quercetin, Staurosporine and derivatives thereof (See Peet and Li, 1999; Phosphorylation of IKB), sodium salicylate (See Ghosh 1994; Yen et al, 1998; BAY-1 17821, BAY 187083 Pierce et al, 1997), Ibuprofen, (See Palayoor et al, 1998), prostaglandin (See Rossi et al, 1997, 2000), and Sulindac (See Yamamoto et al, 1999). The class of compounds termed Go compounds are also such compositions within the scope of the present invention which inhibit the activation of NF-κB. Go compounds are nonglycosidic indollcarbazole and are inhibitors of protein kinase C alpha and beta subspecies. See Qatasha, K. A., Rudolph, C., Marme, D., Schachtele, C., and May, W. S., 1993, Proc. Natl. Acad. Sci. USA, 90:4674-4678, Martiny-Baron, G., Kaniertz, M. G., Blumberg, P. M., Kochs, G., Hug, H., Marmc, D., Schachtele, C. 1993, J. Biol. Chem 268: 9194-9197. One specific Go compound, Go6976, has been shown herein to be an inhibitor of NF-κB activation. Go6976 is designated as C24H18N40. Substances that inhibit the activation of NF-κB, including kinase inhibitors and Go compounds, can be administered by any means known to one skilled in the art, including oral administration, intraperitoneal administration, injective administration, suppository administration, and transdermal administration.

Another embodiment of the present invention is a method for diagnosing patients who would be receptive to treatment with a composition, which, when administered to mammalian subjects with ER− cancer, selectively inhibits activation of NF-κB and results in regression of ER− tumor cell growth in the mammalian subjects. The method comprises obtaining cells from the patient; testing the cells for the presence of estrogen receptor; and testing the cells for the presence of either NF-κB or for the presence of NF-κB activity; wherein the absence in the cells of estrogen receptor in combination with the presence in the cells of either NF-κB or NF-κB activity indicates a patient who would be receptive to treatment.

In this embodiment, compounds such as Go6796 could be used to diagnose ER− cancer. Cells isolated from the breasts of subjects could be cultured in the presence or absence of Go6976. Cells that have their growth rates inhibited in the Go6976 treated cells relative to control would be ER− cancer cells. The subject could then be diagnosed with ER− cancer.

Screening Assays

In yet another embodiment, the present invention is directed to a method for identifying a potential therapeutic agent for use in the treatment of ER− cancers, the method comprising providing ER− cells, tissues, or animals; contacting the ER− cells, tissues, or animals with a composition comprising a candidate substance, wherein the candidate substance inhibits NF-κB activity; and monitoring the progression of the ER− cancer; wherein, if the progression of the ER− cancer is reduced, the candidate substance is identified as a potential therapeutic agent.

The invention also provides a method (also referred to herein as a “screening assay”) for identifying modulators, i.e., candidate or test compounds or agents (e.g., peptides, peptidomimetics, small molecules or other drugs) that have an inhibitory effect on NF-κB activation. The invention also includes compounds identified in the screening assays described herein.

In one embodiment, the invention provides assays for screening candidate or test compounds which inhibit activation of NF-κB directly or indirectly by modulating activities of other members of the NF-κB activation pathway. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12: 145.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37: 1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404-406; Cwirla, et al., 1990. Proc. Natl. Acad. Sci. U.S.A. 87: 6378-6382; Felici, 1991. J. Mol. Biol. 222: 301-310; Ladner, U.S. Pat. No. 5,233,409.).

In one embodiment, an assay is a cell-based assay in which an ER− cancer cell is contacted with a test compound and the ability of the test compound to inhibit activation of NF-κB directly or indirectly and reduce the progression of ER− cancer is determined. The cell, for example, can be of mammalian or human origin, and could be a breast cancer cell. Determining the ability of the test compound to reduce the progression of ER− cancer can be accomplished, for example, by monitoring the progression of the ER− cancer.

The invention also provides a method for monitoring the effectiveness of treatment of a subject with an agent which inhibits activation of NF-κB directly or indirectly (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression or activity of ER− cancer cells in the preadministration sample; (iii) obtaining one or more post-administration samples from the subject; (iv) detecting the level of expression or activity of ER− cancer cells in the post-administration samples; (v) comparing the level of expression or activity of the ER− cancer cells in the pre-administration sample with the ER− cancer cells in the post administration sample or samples; and (vi) altering the administration of the agent to the subject accordingly.

In another embodiment, the present invention is directed to a method of screening for a NF-κB mediated pathology in a tissue, the method comprising isolating the tissue; splitting the tissue into a first portion and a second portion; and culturing the first portion with a compound which inhibits the activation of NF-κB and culturing the second portion without the compound; wherein if the tissue of the first portion decreases in proliferation as compared to the second portion, the tissue has an NF-κB mediated pathology. In this embodiment, the NF-κB mediated pathology can be cancer, more preferably breast cancer, even more preferably ER− breast cancer. Additionally, the tissue can be mammalian tissue, more preferably human. In one specific embodiment of the invention, Go6976 could be the compound used to screen for ER− breast cancer. ER− breast cancer cells would have their growth inhibited while ER+ breast cancer cells would be less affected by the presence of Go6976.

In another embodiment, the present invention is directed to a method of diagnosing the type of cancer cells comprising a tumor in a subject, the method comprising isolating the tumor from the subject; splitting the tumor into a first portion and a second portion; and culturing the first portion with a compound which inhibits the activation of NF-κB and culturing the second portion without the compound, wherein a decrease in proliferation of the tumor of the first portion in relation to the tumor of the second portion diagnoses one type of cancer cell from another type of cancer cell. The types of cancer cells being diagnosed can be estrogen receptor negative breast cancer and estrogen receptor positive breast cancer. In a preferred embodiment, the diagnosis of the cancer cell types is dependent upon differential NF-κB activity.

As disclosed generally herein, and more specifically in the examples, the role of NF-κB on the tumorigenic potential of ER− breast cancer cells was examined in an animal model. The rationale for selecting NF-κB as a therapeutic target was based on (i) the increased level of activated NF-κB observed in many human breast tumors and (ii) on our previous results on its role in enhanced proliferation and cell cycle progression in ER− human breast cancer cells (7).

The requirement of active NF-κB for tumor growth was demonstrated first by blocking its activation with Go6976, a PKC inhibitor, and more specifically by expression of dnIkkβ. Go6976 blocked ER− tumor growth in mice and caused regression of established tumors that could be correlated with the drug's inhibition of NF-κB activation. Anti-cell-proliferation activity of Go6976 maybe caused by decreased NF-κB activation and down-regulation of ccD1 and the subsequent cell cycle progression (7). Although the inhibitory influence of Go6976 on PKCα and −β has been well characterized (28, 29), its influences on other kinases has not been documented. Thus, the inhibition by Go6976 of any one of these other kinases that are involved in the activation of NF-κB, in addition to PKC, is not eliminated.

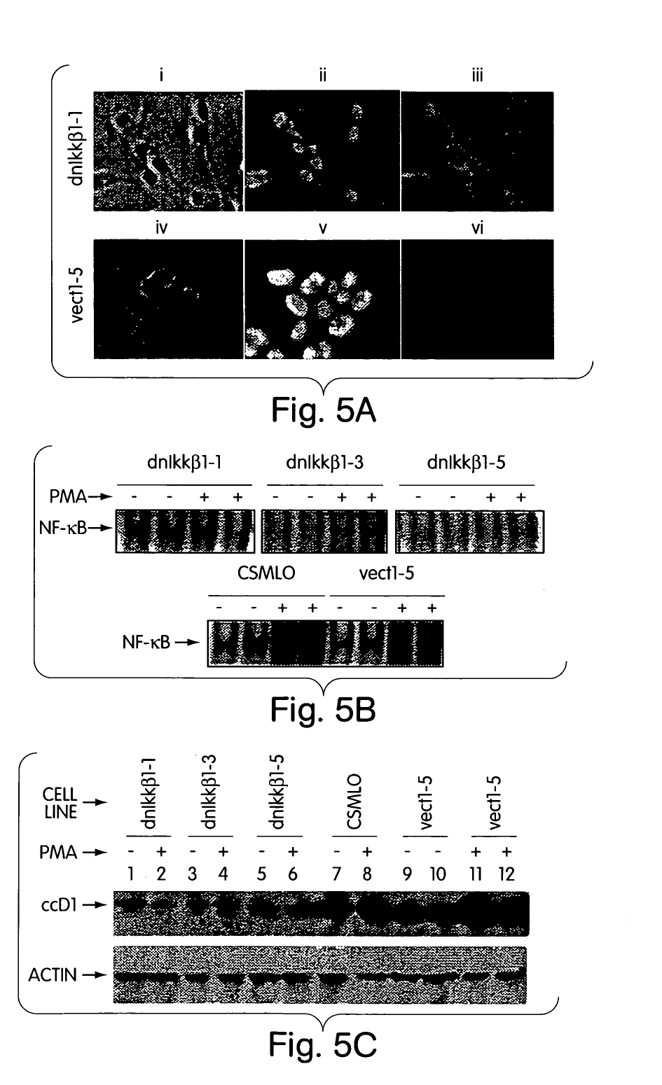

The specific role of active NF-κB for tumor growth in mice was demonstrated with stable expression of dnIkkβ mutant plasmid that selectively blocks NF-κB activation (22, 23) and thereby the downstream event of transactivation of ccD1 (FIG. 5B). Although the growth rate of dnIkkβ-expressing transfectants was reduced by a factor of about 1.25 (doubling time about 60 h) in comparison to the CSMLO cells and vector-control-expressing transfectants (doubling time about 48 h), the vast difference in tumor growth between the former and the latter cannot be attributed only to this retarded cell growth rate. The antitumorigenic effect of NF-κB is a net outcome of its multiple influences on key cellularevents such as apoptosis (14), angiogenesis (35), and cell proliferation (14). Both the pharmacological and genetic manipulations provided support for a direct role of this transcription factor in tumorigenesis by ER breast cancer.

Furthermore, Go6976 caused established tumors to regress rapidly, and their microscopic examination demonstrated DNA clumping and pycnotic cells, suggestive of activation of apoptosis. This drug blocked the kinase-dependent NF-κB activation, but did not affect the basal level of NF-κB. The drug-treated cells maintained limited cell proliferation and induced apoptosis that is consistent with the reported antiapoptotic activity of NF-κB. Thus, Go6976 blocked the antiapoptotic action of NF-κB by selectively blocking its activation in CSMLO cells. Thus, the drug has a dual effect against tumors one is inhibition of NF-κB-mediated cell proliferation, and its more novel activity is to decrease the antiapoptotic activity specific to tumor cells.

The major effect of Go6976 against the tumor was seen without mouse lethality or affecting gross physical damage to the mice or to their vital organs. As its activation is a downstream consequence of the signal initiated by the EGF-EGFR family receptor interaction (7), NF-κB might provide a target for therapy of ER− breast cancer patients with elevated EGFR family receptors. A combination of the current treatment protocol with Herceptin and inhibitors of NF-κB activation, such as Go6976, might provide more effective therapy for this class of human breast cancers. Go6976 might also be useful against the ⅔ of ER− breast cancers that do not express EGFR, and which are not responsive to either Herceptin or classical antihormone treatments. Activation in these cells of NF-κB by means of signal transduction pathways other than that initiated by EGF-EGFR interaction and its role in tumorigenesis may be postulated. For example, in contrast to EGF, transforming growth factor-β (TGF-β) inhibits proliferation of breast cancer cells, and this antiproliferation effect is regulated by the NF-κB/Rel family of transcription factors (36, 37). The observed low TGF-β in ER cells may be associated with the loss of this suppression mediated through TGF-β/Smads signaling for NF-κB/Rel A activation in this type of breast cancer cell (5, 36). NF-κB is also a target of platelet-derived growth factor (PDGF) and this signal is transmitted by the ras/phosphatidylinositol 3-kinase (PI3-kinase)/AKT/Ikk/NF-κB pathway (38).

Toxicity has been one of the major hurdles for defining the role of NF-κB in animals, more specifically those genetically modulated. It is anticipated that inhibition of activation of NF-κB will adversely affect major cellular functions, such as immune responses of B lymphocytes. The up-regulation of this transcription factor in inflammatory diseases is documented, as is the fact that an inhibitor of Ikk reversed inflammatory reactions in an animal model without any adverse effects (27). Similarly, inhibition of the activation of NF-κB by adenoviral delivery of the inhibitory protein IκBα (39) or by inhibition of proteosome-mediated degradation of IκB (40) increased apoptosis and made tumor cells more sensitive to chemotherapy. Our results and the results of other investigators (27, 39, 40) suggest that therapeutic strategies targeted at the inhibition of NF-κB activation are feasible. The residual basal activity of NF-κB remaining after drug treatment may suffice to preserve normal cell viability.

We hypothesize that a basis for the selective toxicity of the drug to ER breast cancer cells (7) is due to their development of an anti-antiapoptotic response. Initially, normal cells are transformed to activate molecular proliferation signals. These signals then also “clash” with normally growth-regulating signals to activate apoptosis (41). Antiapoptotic mutants that permit some cells to survive are then selected during tumor progression; such antiapoptotic mutations as those of p53, Bcl-2, and NF-κB are frequent in advanced cancers. Thus, a basis for the therapeutic index of inhibitors of NF-κB activation is their activity against a specific antiapoptotic function developed in tumor vs. normal cells.

Practice of the invention will be more fully understood from the following examples, which are presented herein for illustration only and should not be considered as limiting the invention in any way.

### EXAMPLES

### Example 1

Inhibition of Tumor Growth by Go6976.

Materials. Mouse mammary adenocarcinoma cells in culture (CSMLO) were grown in complete medium supplemented with 10% FBS and growth factors as described (7, 11, 12). Anti-human ER antibody (SC543), anti-mouse ccD1 antibody, anti-p50, and anti-p65 antibodies for the NF-κB subunits were obtained from Santa Cruz Biotechnology. Rabbit polyclonal IgG raised against the conserved region of actin and anti-Flag (M2) monoclonal antibody were obtained from Sigma-Aldrich. The fluorescein-conjugated goat anti-mouse IgG was from Oncogene Science. Complementary strands of the oligonucleotide (5′-TCGACAGGGACTTTCCGAGAG-3′) containing the NF-κB motif (bold faced) were custom synthesized by Integrated DNA Technologies (Coralville, Iowa). The double-stranded NF-κB-oligonucleotide was end-labeled with [γ-³²P]ATP (NEN) and T4 kinase (New England Biolabs) as described (7), and was used for electrophoretic mobility shift assay (EMSA). Hydrocortisone, insulin, DTT, dimethyl sulfoxide (DMSO), and phenylmethylsulfonyl fluoride were obtained from Sigma. Hybond nitrocellulose membrane and ECL (enhanced chemiluminescence) immunodetection kits were obtained from Amersham Pharmacia. Go6976, a nonglyosidic indolcarbazole, and an inhibitor of protein kinase C alpha and beta was purchased from Calbiochem-Novabiochem (28, 29).

Plasmid constructs. cDNA of the dominant-negative Ikk-β activity mutant k44 M (K M) tagged with amino-terminal Flag sequences was cloned downstream of the cytomegalovirus (CMV) promoter in pCMV5 (22-23). These expression plasmids were provided by Richard B. Gaynor of the University of Texas Southwestern Medical Center, Dallas. For selection of G418-resistant clones the cells were cotransfected with the expression plasmid pcDNA 3.1 (Invitrogen), containing G418-resistant cDNA driven by CMV promoter.

Methods. Measurements of the level of active NF-κB by EMSA. The level of active NF-κB in the nuclear extracts of control and treated cells was determined by its DNA binding activity by EMSA as described 7, 30, 31). The [³²P]DNA-protein complex was identified as a retarded radioactive band in EMSA detected by autoradiography. It was characterized by (i) competition with nonradioactive NF-κB oligonucleotides, (ii) comparative direct binding studies with [γ-³²P]-labeled mutant and wild-type NF-κB-oligonucleotide, and (iii) interaction with antibodies to p65 and anti-p50 subunits, as reported (7).

Isolation of G418-resistant transfectants. To establish stable dominant-negative dnIkkβ-expressing transfectants, the CSMLO cells were transfected with the plasmid K44-M (K M; refs. 22 and 23) along with the selection plasmid pcDNA 3.1 by using Superfect following the protocol provided by the supplier (Qiagen, Chatsworth, Calif.). As a negative control, separate batches of cells were transfected with empty vector (vector control) and the pcDNA 3.1. After 48 h, the transfected cells were harvested and plated into four 100-mm dishes and incubated for another 24 h in complete medium. The transfected cells were then grown for several generations in selection medium (complete medium plus 500 μg/ml G418) and incubated under the standard tissue culture conditions. Six individual clones from K44-M (K M) dnIkkβ-transfected CSMLO cells designated as dnIkkβ1-1 through Ikkβ1-6 and from empty vector control plasmid transfected CSMLO cells designated as vect1-1 through vect1-6 were isolated, maintained in selection medium, and stored in liquid N2. The dnIkk clones containing dnIkkβ-conjugated Flag protein was detected by immunofluorescence technique.

Immunofluorescence detection of Flag-tagged dnIkkβ. Cells (0.5×10⁵) were plated in 8-well chamber slides (Nalge Nunc) and grown in stock medium for 48 h. Cells were then fixed in 4% paraformaldehyde for 10 min followed by incubation in 0.5% Triton X-100 in PBS for 10 min for permeabilization, and were blocked with 10% goat serum in PBS for 1 h. Cells were then incubated for lh in 1:100 diluted solution of anti-Flag-antibody (M2) in 10% goat serum followed by several washes and incubation in 1:100 solution of the secondary antibody fluorescein-goat-anti-mouse IgG (Oncogene) for the detection of Flag conjugated to dnIkk protein. Washed cells were mounted in Vecta Shield (Vector Laboratories) containing 0.1 μg/ml 4′,6-diamidino-2-phenylindole (DAPI) to counter stain the nuclei. To determine the background signal a negative control was performed with the secondary antibody only.

Animal model. The tumorigenic potential of CSMLO and dnIkkβ-expressing stable transfectants was examined in female A-J mice 2-3 weeks old. For each mouse, 1.0 s×10⁶ cells were implanted under the dorsal skin. Palpable tumors were consistently detected within 10-15 days following implantation of the cells. Body weight and tumor volumes (32) were monitored weekly. Go6976 (0.5 ml of 0.05 mM stock solution in 0.1% DMSO) was administered to each animal locally two times a week. The control group received the same volume of the solvent (0.5 ml of 0.1%).

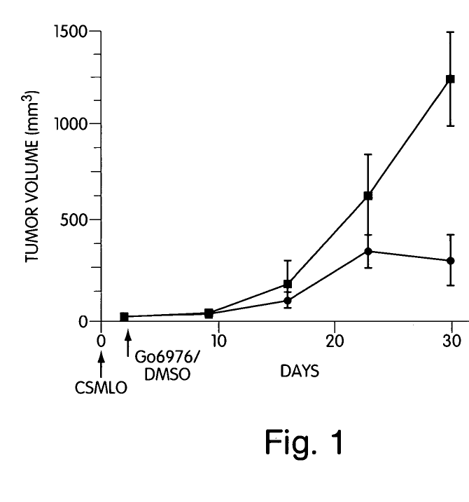

The inhibitory influence of Go6976 on tumor growth was studied by administering Go6976 within 48 h of implantation of CSMLO cells when no palpable tumors were detected. Periodical measurement of tumor volume (32) revealed a substantial inhibition of tumor growth in Go6976-treated mice in comparison to the untreated controls (DMSO-treated group, five animals in each group, FIG. 1). These results demonstrate that Go6976 inhibited the growth of CSMLO-induced tumors when administered within a short period after implantation of the cells.

### Example 2

Regression of Tumor Growth by Go6976.

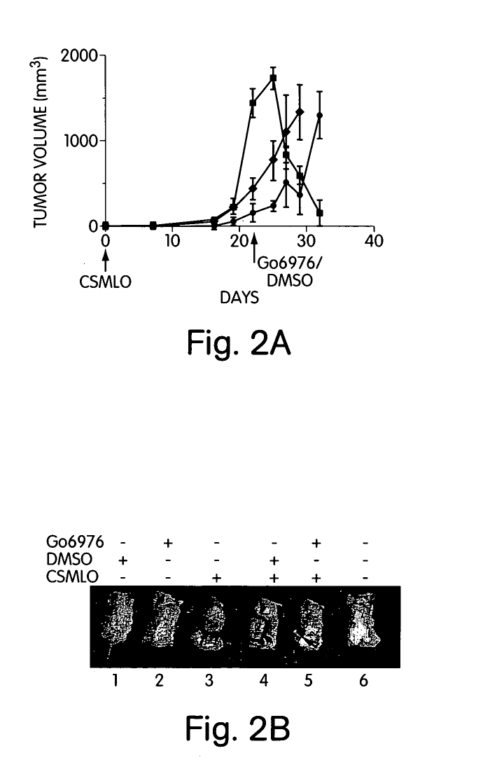

Go6976 not only inhibited tumor growth, but also regression of the full-grown tumors. Tumors in all of five animals treated with Go6976 21 days after implantation of CSMLO cells regressed sharply (FIGS. 2A and B, animal 5). The second group of tumor-bearing mice (five animals) received nothing (FIG. 2 A and B, animal 3) and the third group (five animals) received the solvent DMSO (FIGS. 2A and B, animal 4). The tumor volumes in the second and the third group of animals continued to increase (FIGS. 2A and B, animals 3 and 4). Mice without tumor treated either with DMSO or with Go6976 (FIGS. 2B, animals 1 and 2) did not show any apparent physical defects as judged by body weight and agility, and these parameters were not different from the animals without any treatments (FIG. 2, animal 6).

### Example 3

Inhibition of PMA-Induced Activation of NF-κB by Go6976 in CSMLO Cells.

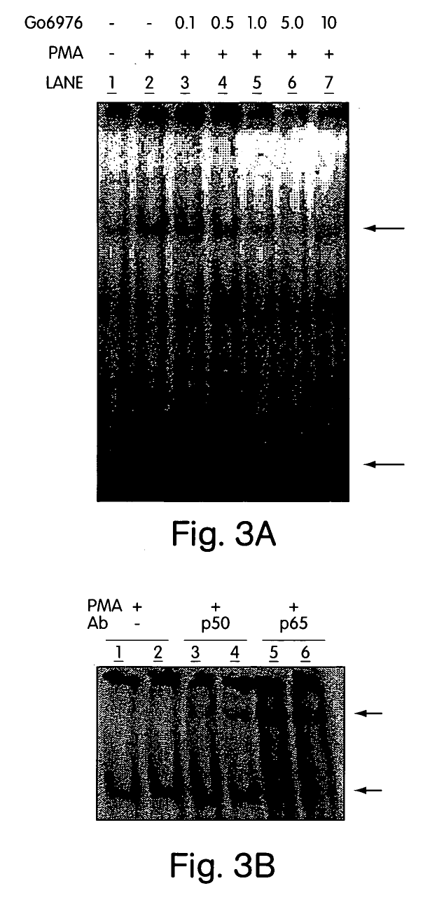

The previously reported NF-κB inhibitory effect of Go6976 in human breast cancer (7) and Jurkat T lymphocytic (33) cell lines is confirmed also in CSMLO cells. CSMLO cells are ER as determined by Western blot analysis. A low basal level activity of NF-κB was detected in nuclear extracts from CSMLO cells as determined by EMSA (FIG. 3A, lane 1), which was stimulated by PMA (20 ng/ml; lane 2). Activation could be detected at 2-4 h of PMA treatment, was maximal at 6 h, and remained the same for 24 h (data not shown). By simultaneous treatment with Go6976 for 48 h, the PMA-induced elevation of NF-κB activation was inhibited in a concentration-dependent manner (lanes 3-7). The growth of cells under culture conditions was not affected by this short-term drug treatment with 10 μM Go6976 (7). The retarded radioactive DNA-protein complex was supershifted with either anti-p50 (FIG. 3B, lanes 3 and 4) or anti-p65 (lanes 5 and 6) antibody, but not with anti-cRel antibody (data not shown), thus characterizing the active NF-κB in CSMLO cells as a p50/p65 heterodimer.

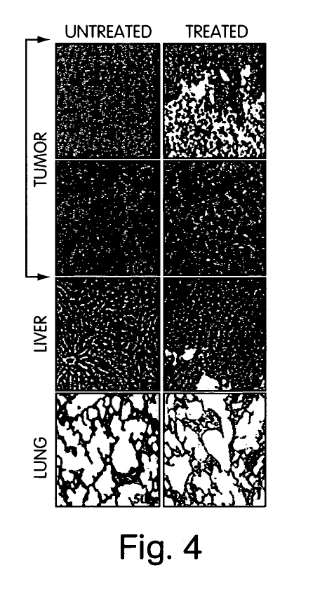

FIG. 4 shows histological changes in tumor, liver, and lung tissues from untreated (Left) and Go6976-treated animals (Right). Tumor tissues from untreated and DMSO-treated (data not shown) animals demonstrated characteristic tumor cell phenotypes, such as enlarged nuclei and increased mitotic index (indicated by arrowheads). In contrast, in Go6976-treated animals extensive necrosis (indicated by N) of the tumor tissues was detected with few residual tumor cells (indicated by T). Similar histological examination of liver and lung tissue (FIG. 4) sections from tumor bearing animals (FIG. 2B, animals 3, 4, and 5) or non-tumor-bearing animals (data not shown) did not show any microscopically detectable pathological changes. Because CSMLO cells are characterized as noninvasive, these observations were expected (11, 12). Examination of residual tumor tissues in treated animal at higher magnification (100×) revealed a large number of pycnotic cells with nuclear clumping suggestive of DNA fragmentation and apoptosis (indicated by stars in Right), whereas this was not observed in tumor tissues from untreated animals (Left). These results demonstrated that localized Go6976 treatment selectively killed the target tumor cells at a concentration not toxic to the vital organs, liver, and lung. These results (FIGS. 1 and 2) and prior data (7) suggest that Go6976 is a selective killer of ER mouse mammary epithelial tumor cells.

### Example 4

Selective Inhibition of NF-κB Activation by Stable Expression of Dominant-Negative Ikk-β(dnIKK β) Mutants.

Our second experimental approach for examining the role of NF-κB in the CSMLO cell-induced tumorigenesis was to selectively block activation of the NF-κB-IκB complex and thereby retain it in its inactive state in the cytoplasm. This was accomplished by the stable expression of a dominant-negative mutant of one of the subunits of Ikk. Because Ikk-β is more potent in activating NF-κB than is Ikk-α, we focused our initial studies on dnIkkβ-expressing transfectants and compared them with parental CSMLO cells and transfectants expressing the vector control plasmid. The dnIkkβ-expressing transfectants grew comparatively slower than the parent CSMLO cells.

### Example 5

Immunofluorescent Detection of Flag-Tagged dnIkkβ in Transfectants.

The dnIkkβ transfectants were characterized by identifying the Flag protein tagged to it by an immunofluorescence technique using Flag-specific antibody. FIG. 5A demonstrates the immunofluorescence signal of one dnIkkβ transfectant (Upper, iii) that represents the exogenously introduced Flag-tagged dominant-negative mutant dnIkkβ protein in the cytoplasm. A G418 resistant transfectant with empty vector plasmid did not show this fluorescence (Lower, vi), suggesting that the positive signal observed in this transfectant is specific for Flag-tagged dnIkkβ protein.

### Example 6

The Level of Active NF-κB in dnIkkβ-Expressing Transfectants.

The functional state of NF-κB in the dnIkkβ-expressing transfectants was established by measuring its DNA-binding activity with EMSA. The basal level of active NF-κB was not further stimulated by PMA in three different clones of dnIkkβ-expressing transfectants as judged by its unaltered [³² P]DNA binding activity (FIG. 5B Upper). In contrast, the basal level of active NF-κB was strongly elevated by PMA in parent CSMLO cells and in transfectants with vector control plasmid vect-1-5 (FIG. 5B Lower). These results demonstrated that PMA activation above the basal level of NF-κB is blocked by externally introduced dnIkkβ, thereby establishing the experimental goal of selective inhibition of NF-κB activation by specifically targeting the Ikk with dnIkkβ.

### Example 7

The Level of ccD1 in dnIkkβ-Expressing Transfectants.

Up-regulation of the ccD1 is a downstream consequence of NF-κB activation (7, 34). Similar to the results obtained in human breast cancer cells (7), Go6976 also blocked transactivation of ccD1 in CSMLO cells transfected with dnIkkβ. The elevated level of ccD1 following PMA treatment measures activation of the NF-κB -IκB complex. FIG. 5C demonstrates that ccD1 was not altered by PMA treatment of the three clones of dnIkkβ-expressing transfectants (5C, lanes 1-6) in which NF-κB activation was effectively blocked (5B, Upper), whereas ccD1 level was substantially elevated by PMA in parental and transfectants carrying the control plasmid vect-1-5 (5C, lanes 7-12). These levels of ccD1 in CSMLO cells and transfectants correlates well with activation of NF-κB-IκB complex (FIG. 5B). The basal level of ccD1 in the dnIkkβ-expressing transfectants was lower than that in the parent and vector-control-expressing cells. This may be a reflection of the short half-life of ccD1. Thus, ability to up-regulate downstream ccD1 gene expression following PMA treatment in the parent CSMLO and in transfectant expressing vector control plasmid, and the inability to do the same in dnIkkβ-expressing transfectants can be correlated to the level of activation of inactive NF-κB-IκB complex in these cells.

### Example 8

Tumorigenic Potential of CSMLO Cell-Expressing dnIkkβ Mutants.

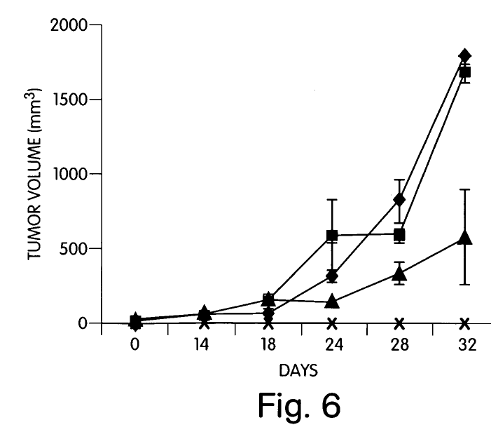

The consequence of blocked NF-κB activation in dnIkkβ-expressing transfectants on tumor growth was examined in the mouse tumor model. FIG. 6 shows volumes of tumors induced by CSMLO cells (five animals), two transfectants with vector control plasmid (six animals, three per clone), and two transfectants expressing dnIkkβ (six animals, three per clone). The CSMLO cells and transfectants carrying the vector control plasmid generated tumors similarly. One dnIkkβ-expressing clone (dnIkkβ1-1) did not form tumor in any of the three animals even after 32 days. The other dnIkkβ-expressing clone (dnIkkβ1-3) formed tumors at a substantially reduced rate and of smaller sizes compared with those formed by the CSMLO or the vector control plasmid expressing transfectants (FIG. 6). The loss or decreased tumorigenic potential of the dnIkkβ-expressing transfectants, compared with parent and the vector controls was in agreement with the reduced level of NF-κB activation and cellular level of ccD1. All of these results, especially those with dnIkkβ expression that selectively blocked NF-κB-IκB activation and ccD1 transactivation, strongly suggest a role of NF-κB in tumorigenesis in ER mammary epithelial cells.

The above-described results show clearly that compounds which block NF-κB activation, such as Go6796, can be used to diagnose and treat ER− mammary epithelial cell-mediated tumorigenesis. These results show that blocking NF-κB activation not only inhibits cell proliferation, but also antagonizes the antiapoptotic role of NF-κB in ER− breast cancer cells. The present invention thus provides compositions and methods for screening, diagnosing, or treating an NF-κB induced pathology through the use of NF-κB modulating agents. More specifically, it relates to methods for inhibiting growth of ER− breast cancer cells by administering to the cells a composition which reduces NF-κB activity.

Other Embodiments

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. Other aspects, advantages, and modifications are considered to be within the scope of the following claims. The claims presented are representative of the inventions disclosed herein. Other, unclaimed inventions are also contemplated. Applicants reserve the right to pursue such inventions in later claims.

References:

1. Jordan, V. C. (1995) Breast Cancer Res. Treat. 36, 267-285.

2. Hedden, A., Muller, V. & Jensen, E. V. (1995) Ann. N.Y. Acad. Sci. 761, 109-120.

3. Nakshatri, H., Bhat-Nakshatri, P., Martin, D. A., Goulet, R. J., Jr. & Sledge, G. W. (1997) Mol. Cell. Biol. 17, 3629-3639.

4. Bhat-Nakshatri, P., Newton, T. R., Goulet, R., Jr. & Nakshatri, H. (1998) Proc. Natl. Acad. Sci. USA 95, 6971-6976.

5. Sovak, M. A., Bellas, R. E., Kim, D. W., Zaneiski, G. J., Rogers, A. E., Traish, A. M. & Sonenshein, G. E. (1997) J. Clin. Invest. 100, 2952-2960.

6. Rayet, B. & Gelinas, C. (1999) Oncogene 18, 6938-6947.

7. Biswas, D. K., Cruz, A. P., Gansberger, E. & Pardee, A. B. (2000) Proc. Natl. Acad. Sci. USA 97, 8542-8547.

8. Sun, L. & Carpenter, G. (1998) Oncogene 16, 2095-2102.

9. Eithier, S. P. (1995) J. Natl. Cancer Inst. 87, 964-973.

10. Galang, C. K., Garcia-Ramirez, J. J., Solski, P. A., Westwick, J. K., Der, C. J., Neznanov, N. N., Oshima, R. G. & Hauser, C. A. (1996) J. Biol. Chem. 271, 7992-7998.

11. Senin, V. M., Buntsevich, A. M., Afanasyeva, A. V. & Kiseleva, N. S. (1983) Exp. Oncol. USSR 5, 35-38.

12. Ebralidze, A., Tulchinsky, E., Grigorian, M. S., Afanasyeva, A., Senin, V., Revazova, E. & Lukanidin, E. (1989) Genes Dev. 3, 1086-1093.

13. Baeurle, P. A. & Baltimore, D. (1996) Cell 87, 13-20.

14. Baldwin, A. S. (1996) Annu. Rev. Immunol. 14, 649-681.

15. Pahl, H. L. (1999) Oncogene 18, 6853-6866.

16. Sen, R. & Baltimore, D. (1986) Cell 46, 705-716.

17. Verma, I. M., Stevensen, J. K., Schwarz, E. M., VanAntwarp, D. & Miyamoto, S. (1995) Genes Dev. 9, 2723-2735.

18. Karin, M. (1999) Oncogene 18, 6867-6874.

19. Rothwarf, D. M., Zandi, E., Natoli, G. & Karin, M. (1998) Nature (London) 395, 297-300.

20. Yamaoka, S., Courtuis, G., Bessia, C., Whiteside, S. T., Weil, R., Agon, F., Kirk, H. E., Kay, R. J. & Israel, A. (1998) Cell 93, 1231-1240.

21. Mercurio, F., Murry, B. W., Shevchenko, A., Bennett, B. L., Young, D. B., Li, J. W., Pascual, G., Motiwala, A., Zhu, H., Mann, M. & Manning, A. M. (1999) Mol. Cell. Biol. 19, 1526-1538.

22. Yin, M. -J., Christenson, L. B., Yamamoto, Y., Kwak, Y. -T., Xu, S., Mercurio, F., Burlossa, M., Cobb, M. H. & Gaynor, R. B. (1998) Cell 93, 875-884.

23. Yin, M. -J., Yamamoto, Y. & Gaynor, R. B. (1998) Nature (London) 396, 77-80.

24. Li, Q., Estepa, G., Memet, S., Israel, A. & Verma, I. M. (2000) Genes Dev. 14, 1729-1733.

25. Li, Q., Lu, Q., Hwang, J. Y., Buscher, D., Lee, K. F., Izpisua-Belmonte, I. C. & Verma, I. M. (1999) Genes Dev. 13, 1322-1328.

26. Li, Q., Van Antwarp, D., Mercurio, F., Lee, K. F. & Verma, I. M. (1999) Science 284, 321-325.

27. May, M. J., D'Acquisto, F., Madge, L. A., Glockner, J., Pober, J. S. & Ghosh, S. (2000) Science 289, 1550-1553.

28. Qatasha, K. A., Rudolph, C., Marine, D., Schachtele, C. & May, W. S. (1993) Proc. Natl. Acad. Sci. USA 90, 4674-4678.

29. Martiny-Bbaron, G., Kazaniertz, M. G., Blumberg, P. M., Kochs, G., Hug, H., Marine, D. & Schachtele, C. (1993) J. Biol. Chem. 268, 9194-9197.

30. Dignam, J. D., Lebovitz, R. M. & Roeder, R. D. (1983) Nucleic Acids Res. 11, 1475-1489.

31. Biswas, D. K., Reddy, P. M., Pickard, M., Makkad, M., Pettit, N. & Pardee, A. B. (1998) J. Biol. Chem. 273, 33817-33824.

32. Grigorian, M., Ambartsumian, N., Lykkesfeldt, A. E., Bastholm, L., Elling, F., Georgiev, G. & Lukanidin, E. (1996) Int. J. Cancer 67, 831-841.

33. Biswas, D. K., Ahlers, C. M., Dezube, B. J. & Pardee, A. B. (1993) Proc. Natl. Acad. Sci. USA 90, 11044-11048.

34. Hinz, M., Krappmann, D. A., Eichten, A., Heder, C., Scheidreit, C. & Strauss, M. (1999) Mol. Cell. Biol. 20, 1448-1459.

35. Dikov, M. M., Oyama, T., Cheng, P., Takashi, T., Takahashi, K., Sepetavec, T., Edwards, B., Adachi, Y., Nadaf, S., Daniel, T. & Carbon, D. P. (2001) Cancer Res. 61, 2015-2021.

36. Sovak, M. A., Arsura, M., Zanieski, G., Kavanagh, K. T. & Soenenshein, G. E. (1999) Cell Growth Differ. 10, 537-544.

37. Lopez-Rovira, T., Chjalaux, E., Rosa, J. L., Bartrons, R. & Ventura, F. (2000) J. Biol. Chem. 275, 28937-28946.

38. Ramashokova, J. A. & Makarov, S. S. (1999) Nature (London) 401, 86-90.

39. Wang, C. -Y., Cusack, J. C., Liu, R. & Baldwin, A. S., Jr. (1999) Nat. Med. 5, 412-417.

40. Cusack, J. C., Jr., Liu, R., Houston, M., Abendroth, K., Elliott, P. J., Adams, J. & Baldwin, A. S., Jr. (2001) Cancer Res. 61, 3535-3540.

41. Evan, G. & Littlewood, T. (1998) Science 281, 1317-1322.

We claim

1. A composition, which, when administered to mammalian subjects with ER− cancer, selectively inhibits activation of NF-κB and results in regression of ER− tumor cell growth in the mammalian subjects.

2. A pharmaceutical composition comprising the composition of claim 1 and a pharmaceutically acceptable carrier.

3. A kit comprising in one or more containers the pharmaceutical composition of claim 2.

4. A method for inhibiting growth of ER− cancer cells, the method comprising administering to the cells a composition which reduces NF-κB activity in the cell in an amount sufficient to inhibit growth of the ER− cancer cells.

5. The method of claim 4, carried out on human cells.

6. The method of claim 4, carried out in vitro.

7. The method of claim 4, carried out in vivo.

8. The method of claim 1, wherein the ER− cancer is ER− breast cancer.

9. A method for diagnosing and treating mammalian cancers in a subject, the method comprising:
(a) obtaining cancer cells from the subject;
(b) testing the cancer cells from the subject for the presence of estrogen receptor;
(c) diagnosing the mammalian cancer as ER+ if estrogen receptor is present in the cells or as ER− if estrogen receptor is absent from the cells; and
(d) administering to the subject diagnosed with ER− cancer a composition which reduces NF-KB activity, in an amount sufficient to inhibit growth of ER− cancer cells.

10. The method of claim 9, where the composition is a kinase inhibitor.

11. The method of claim 10, where the kinase inhibitor is a Go compound.

12. The method of claim 11, where the Go compound is Go6796.

13 The method of claim 9, wherein the ER− cancer cells are ER− breast cancer cells.

14. A method for treating ER− cancers in a mammalian subject, the method comprising administering to the mammal a composition which reduces NF-κB activity and monitoring the mammal to determine the state of the cancer, wherein the composition is administered in an amount sufficient to inhibit the growth of ER− cancer cells.

15. The method of claim 14, wherein the composition is a kinase inhibitor.

16. The method of claim 15, wherein the kinase inhibitor is a Go compound.

17. The method of claim 16, wherein the Go compound is Go6796.

18. The method of claim 14, wherein the ER− cancer is ER− breast cancer.

19. A method of treating an NF-κB mediated pathology in a subject wherein the pathology is treated by administration of Go6976 to the subject.

20. The method of claim 19, wherein the pathology mediated is a cancer.

21. The method of claim 20, wherein the cancer is breast cancer.

22. The method of claim 21, wherein the breast cancer is comprised of estrogen receptor negative breast cancer cells.

23. The method of claim 19, wherein the subject is a mammal.

24. The method of claim 19, wherein the subject is a human.

25. The method of claim 19, wherein the administration of Go6976 to the subject is performed by a method that is a member of the group consisting of oral administration, intraperitoneal administration, injective administration, suppository administration, and transdermal administration.

26. A method for diagnosing patients who would be receptive to treatment with the composition of claim 1, the method comprising:
(a) obtaining cells from the patient;
(b) testing the cells for the presence of estrogen receptor;
(c) testing the cells for the presence of either NF-κB or for the presence of NF-κB activity;
wherein the absence in the cells of estrogen receptor in combination with the presence in the cells of either NF-κB or NF-κB activity indicates a patient who would be receptive to treatment.

27. A method for identifying a potential therapeutic agent for use in the treatment of ER− breast cancers, the method comprising:
(a) providing ER− cells, tissues, or animals;
(b) contacting the ER− cells, tissues, or animals with a composition comprising a candidate substance, wherein the candidate substance inhibits NF-κB activity; and
(c) monitoring the progression of the ER− cancer;
wherein, if the progression of the ER− cancer is reduced, the candidate substance is identified as a potential therapeutic agent.

28. A method of screening for a NF-κB mediated pathology in a tissue, the method comprising:
(a) isolating the tissue;
(b) splitting the tissue into a first portion and a second portion; and
(c) culturing the first portion with a compound which inhibits the activation of NF-κB and culturing the second portion without the compound;
wherein if the tissue of the first portion decreases in proliferation as compared to the second portion, the tissue has an NF-κB mediated pathology.

29. The method of claim 28, wherein the tissue is a mammalian tissue.

30. The method of claim 29, wherein the tissue is a human tissue.

31. The method of claim 28, wherein the NF-κB mediated pathology is breast cancer.

32. The method of claim 31, wherein the NF-κB mediated pathology is ER− breast cancer.

33. The method of claim 28, wherein the compound is a kinase inhibitor.

34. The method of claim 33, wherein the compound is Go6796.

35. A method of diagnosing the type of cancer cells comprising a tumor in a subject, the method comprising:
(a) isolating the tumor from the subject;
(b) splitting the tumor into a first portion and a second portion; and
(c) culturing the first portion with a compound which inhibits the activation of NF-κB and culturing the second portion without the compound,
wherein a decrease in proliferation of the tumor of the first portion in relation to the tumor of the second portion diagnoses one type of cancer cell from another type of cancer cell.

36. The method of claim 35, wherein the tumor is a breast cancer tumor.

37. The method of claim 36, wherein the types of cancer cells being diagnosed are estrogen receptor negative breast cancer and estrogen receptor positive breast cancer.
